(19) United States
(12) Patent Application Publication
(10) Pub. No.: US 2017/0218330 A1
(43) Pub. Date: Aug. 3, 2017

(54) SYSTEMS AND METHODS FOR THE SEPARATION OF CELLS FROM MICROCARRIERS USING A SPINNING MEMBRANE

(71) Applicant: Fenwal, Inc., Lake Zurich, IL (US)

(72) Inventors: Christopher Wegener, Libertyville, IL (US); Kyungyoon Min, Kildeer, IL (US)

(21) Appl. No.: 15/421,075

(22) Filed: Jan. 31, 2017

Related U.S. Application Data
(60) Provisional application No. 62/289,677, filed on Feb. 1, 2016.

(51) Int. Cl. C12N 5/00; C12M 1/34; C12M 1/26; C12M 3/04; C12M 1/00

(52) U.S. Cl. CPC C12N 5/0075; C12M 27/10; C12M 29/04; C12M 33/14; C12M 41/44; C12N 2531/00

(57) ABSTRACT

Methods and systems for processing suspensions of biological cells and microcarriers are disclosed. The biological cells are separated from the microcarriers by introducing the suspension into a spinning membrane separator whereby the biological cells pass through the membrane and the microcarriers do not pass through the membrane.

### FIELD OF THE DISCLOSURE

The present disclosure is directed to the methods and systems for processing a suspension of biological cells and microcarriers to separate the cells from the microcarriers. More particularly, the present disclosure is directed to methods and systems for processing the suspension and achieving the separation utilizing a separation device that includes a spinning membrane.

### BACKGROUND

Cell therapies represent an emerging field of medical research. A cell therapy procedure often involves the manipulation of autologous or allogeneic cells for a particular patient or indication. Often, one of these manipulation steps requires in vitro culture/expansion of adherent cell lines. Traditionally, these adherent cells have been grown in culture media in 2D culture vessels, such as T-Flasks or Petri Dishes.

In many applications, the production of as many cells as possible allows for reduction of cost and/or more efficacious treatments. Traditional 2D culture vessels often do not provide enough surface area to accommodate the desired number of cells. One way of providing additional surface area is to grow adherent cells on the surface of plastic microcarriers, small particles that have an affinity for growing cells thereon. Growing cells in this fashion often dramatically increases the number of cells available for further processing and for cell therapy procedures.

Once grown, these cells must be harvested so that they can be reinfused into a patient. Where cells have been grown on a microcarrier, a cleaving agent is often added (e.g., Trypsin or a synthetic alternative) to the culture to separate the grown cells from the microcarriers and allow for resuspension of the now separated cells. Further isolation of the cells or harvesting can be achieved through simple filtration where the larger diameter microcarriers are captured by the filter and the biological cells pass through the membrane. However, where large volumes of the culture are harvested, “dead-end” filtration, i.e., filtration with a single inlet, single outlet and perpendicular flow across the membrane is often inefficient and expensive. Traditional filtration methods may result in a reduction of filtrate flow due to the accumulation of microcarriers on the filter surface. Thus, traditional filtration often requires filters with large surface areas and high up-stream volumes to accomodate the volume of the retained microcarriers.

Accordingly, it would be desirable to provide methods and systems whereby large volumes of suspensions including biological cells and microcarriers can be separated whereby the separated microcarriers are removed and the biological cells are collected for further processing, without the drawbacks of traditional filtration systems.

### SUMMARY

In one aspect, the present disclosure is directed to a method for separating biological cells from microcarriers in a suspension. The method includes introducing a suspension of biological cells and microcarriers into a separation device with a relatively rotatable cylindrical housing and an internal member. The cylindrical housing has an interior surface and the internal member has an exterior surface. The surfaces define a gap therebetween, wherein one of the surfaces includes a porous membrane with pores sized to retain the microcarriers while allowing the biological cells to pass through the membrane. The method further includes withdrawing the separated biological cells from the separation device.

In another aspect, the present disclosure is directed to a system for separating biological cells from microcarriers in a suspension that includes a reusable hardware unit with a separation device drive unit for receiving a separation device. The system further includes a disposable fluid circuit mountable on the reusable hardware unit. The disposable fluid circuit includes a separation device with a relatively rotatable cylindrical housing and an internal member, wherein the cylindrical housing has an interior surface and said internal member has an exterior surface, the surfaces defining a gap therebetween. One of the surfaces includes a porous membrane with pores sized to retain the microcarriers while allowing the biological cells to pass through the membrane. The separation device includes an inlet and at least one outlet in fluid communication with the gap. The system also includes a controller configured and/or programmed to control the operation of the separation device.

### BRIEF DESCRIPTION OF THE DRAWINGS

FIG. 1 is a schematic view of one embodiment of a disposable fluid circuit useful in the systems and methods described herein;

FIG. 2 is an enlarged view of the front panel of the reusable processing apparatus;

FIG. 3 is a perspective view, partially broken away, of the separation device of FIG. 1;

FIG. 4 is another view of the front panel of a reusable processing and/or cell washing apparatus with a disposable fluid circuit mounted thereon;

FIG. 5(a) is a schematic view of a suspension of biological cells and microcarriers undergoing separation inside the separation device;

FIG. 5(b) is a schematic view of a suspension of biological cells and microcarriers undergoing cleaving and separation inside the separation device; and

FIG. 6 is a flow chart setting forth steps of the method disclosed herein.

### DETAILED DESCRIPTION OF THE EMBODIMENTS

A description of cell culturing or the process of growing cells on microcarriers is beyond the scope of the present application. Those skilled in the art will recognize the bioreactor systems used to grow the desired cells. such as, but not limited to, GE Healthcare Xuri, PBS Biotech Bioreactor Systems, or Eppendorf/New Brunswick Bio BLU Systems. When the desired cells have achieved sufficient growth in the bioreactor system, the cell/microcarrier aggregates may be drained into a container for subsequent addition of the cleaving agent. Once the cells have been cleaved from the microcarrier substrates and the now separated microcarriers and cells are collected in a container, the container may be directly connected to the system described herein or to a source container that is part of the system. Alternatively, as described below, a source of grown cells still attached to the microcarriers may be directly connected to the system disclosed herein or to a source container that is integrally connected to the system. The uncleaved cell/carrier aggregates may be combined with a cleaving agent in a container that is integrally connected to the system. After a sufficient period of time to effect cleaving, the now cleaved cells and microcarriers are introduced into the system. This way, both the cleaving and separation steps may be performed by the system herein disclosed.

The methods and systems disclosed herein typically employ a reusable separation apparatus and one or more disposable processing circuits adapted for association with the reusable apparatus. The reusable separation apparatus may be any apparatus that can provide for the automated processing of biological cells. By “automated,” it is meant that the apparatus can be pre-programmed to carry out the processing steps of a biological fluid processing method without substantial operator involvement. Of course, even in the automated system of the present disclosure, it will be understood that some operator involvement will be required, including the loading or mounting of the disposable fluid circuits onto the reusable apparatus and entering processing parameters. Additional manual steps may be required as well. However, the reusable apparatus can be programmed to perform the cleaving of cells from the microcarriers and the processing of the biological cells and microcarriers through the disposable circuit(s) described below without substantial operator intervention.

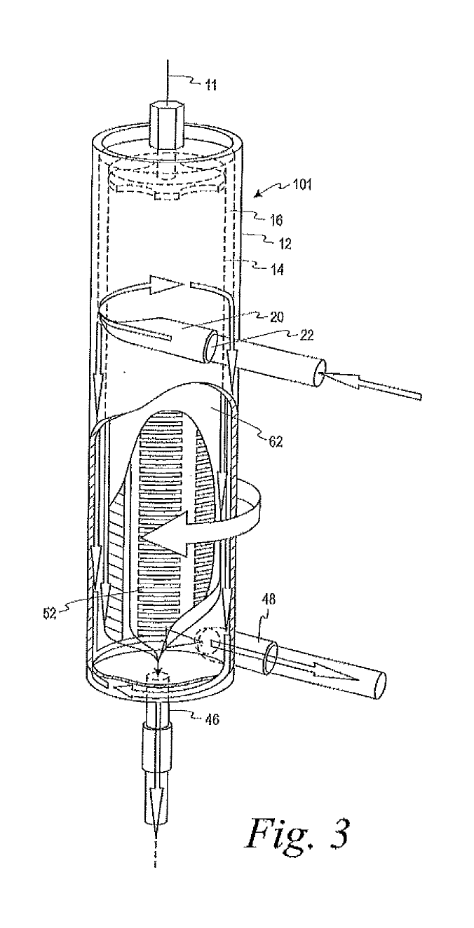

The reusable processing apparatus is capable of effecting the separation of biological cells from particles, such as microcarriers or other synthetic substrates. Thus, the reusable apparatus may generate conditions which allow for the separation of biological cells from such particles. In accordance with the present disclosure, one preferred means for separating biological cells from the particles or substrates is an apparatus that uses a spinning porous membrane to separate one component from other components. An example of such apparatus is the Autopheresis C® sold by Fenwal, Inc. of Lake Zurich, Ill. A detailed description of a spinning membrane may be found in U.S. Pat. No. 5,194,145 to Schoendorfer, which is incorporated by reference herein in its entirety, and in International (PCT) Application No. PCT/US2012/028492, filed Mar. 9, 2012, the contents of which is also incorporated herein in its entirety. In addition, systems and methods that utilize a spinning porous membrane are also disclosed in U.S. Provisional Patent Application No. 61/537,856, filed on Sep. 22, 2011, International (PCT) Application No. PCT/US2012/028522, filed Mar. 9, 2012, International (PCT) Application No. PCT/US2012/054859, filed Sep. 12, 2012 and U.S. patent application Ser. No. 14/574,539 filed Dec. 18, 2014, the contents of each are incorporated herein by reference. The references identified above describe a membrane covered spinner having an interior collection system disposed within a stationary shell. While a detailed discussion of the separation device is beyond the scope of this application, the spinning membrane separation device is shown in FIG. 3 and is briefly discussed below.

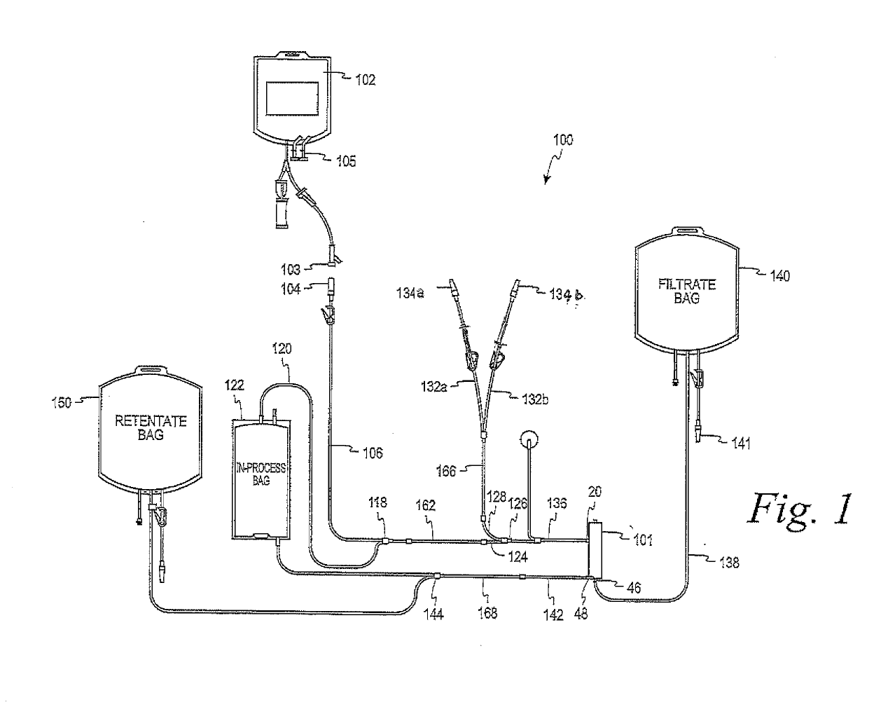

Turning first to FIG. 1, the systems described herein preferably include a disposable fluid circuit for use in the processing and separation of the suspension of biological cells and microcarriers. Fluid circuit 100 is adopted for mounting onto the reusable hardware component, described below. Circuit 100 may include an integrated separation device, such as, but not limited to, the spinning membrane 101 described herein. Circuit 100 may also include filtrate bag or container 140, retentate container or bag 150, and in-process container 122. Disposable fluid circuits of the type described below may further include sampling assemblies (not shown) for collecting samples of biological cells, “final” cell product, or other intermediate products obtained during the biological fluid processing.

As will be seen in the Figures and described in greater detail below, the disposable fluid processing circuits include tubing that defines flow paths throughout the circuit, as well as access sites for sterile or other connection to containers of processing solutions, such as wash solutions, treating (e.g., cleaving) agents, and sources of the biological cell and microparticle suspension fluid. As will be apparent from the disclosure herein, source containers may be attached in sterile fashion to the circuit 100.

As shown in FIG. 1, the tubing of circuit 100 includes spaced tubing segments identified by reference numerals 162, 166, 168. The tubing segments are provided for mating engagement with the peristaltic pumps of the reusable hardware apparatus 200 discussed below. The containers and the plastic tubing are made of conventional medical grade plastic that can be sterilized by sterilization techniques commonly used in the medical field, such as, but not limited to, radiation or autoclaving. Plastic materials useful in the manufacture of containers and tubing in the circuits disclosed herein include plasticized polyvinyl chloride. Other useful materials include acrylics. In addition, certain polyolefins may also be used.

The biological cell/particle (microcarrier) suspension to be processed is typically provided in a source container 102, shown in FIG. 1 as (initially) not connected to the disposable set. As noted above, source container 102 may be attached (in sterile fashion) at the time of use. Source container 102 has one or more access sites 103, 105, one of which may be adapted for (sterile) connection to fluid circuit 100 at docking site 104. Preferably, source containers may be attached in a sterile manner by employing sterile docking devices, such as the BioWelder, available from Sartorius AG, or the SCD IIB Tubing Welder, available from Terumo Medical Corporation. A second access port 105 may also be provided for extracting fluid from the source container 102.

In another embodiment, source container 102 may be pre-attached to circuit 100. In such embodiment, the biological cell/particle suspension may be transferred, in sterile fashion, from a container that is used to grow the cells on the microcarriers.

With further reference to FIG. 1, tubing 106 is connected to downstream branched-connector 118. Branched-connector 118 communicates with tubing 106 and tubing 120, which provides a fluid flow path from “in-process” container 122, described in greater detail below. Tubing segment 124 extends from branched-connector 118 and is joined to a port of further downstream branched-connector 126. A separate flow path defined by tubing 128 is also connected to a port of branched-connector 126.

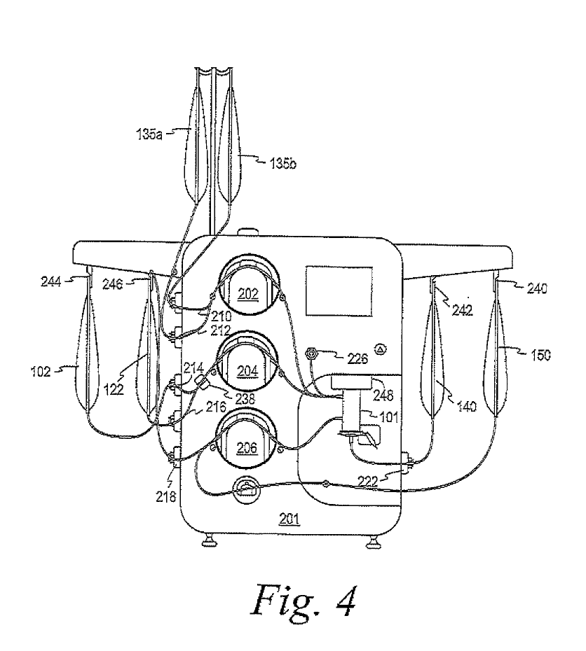

In accordance with the fluid circuit of FIG. 1, a container of wash or other processing/treating solution may be attached (or pre-attached) to set 100. As shown in FIG. 1, tubing 132a (defining a flow path) preferably includes and terminates in an access site such as spike connector 134a. Access site 134a is provided to establish flow communication with a container 135 (shown in FIG. 4) of a wash fluid, such as saline or other solution. More preferably, flow communication between tubing 132a and a container of wash solution may be achieved by sterile connection device, such as, but not limited to, the previously mentioned Terumo SCD IIB. The wash medium or fluid flows from the wash fluid source through tubing segment 132a, and then passes through tubing 128 to the input of the branched-connector 126 described above.

Additional access sites such as site 134b may also be provided. Such additional access sites may be used to establish fluid communication with other solutions and/or agents (e.g., bag 135b shown in FIG. 4). For example, in one embodiment, access site 134a or 134b may be used to establish fluid communication with a container including a cleaving agent for separating the biological cells from the surface of the microcarriers. A container of a cleaving agent neutralizing solution may also be connected to circuit 100. In short, it will be understood that solutions such as one or more of a wash solution, a cleaving agent and a cleaving agent neutralizing solution may be attached to fluid circuit at access sites 134a, 134b and additional access sites, as necessary.

As shown in FIG. 1, tubing segment 136 defines a flow path connected at one end to branched-connector 126 and to an inlet port 20 of the separator 101. Preferably, in accordance with the present disclosure, separation device 101 is a spinning membrane separator of the type described in U.S. Pat. No. 5,194,145 and U.S. Pat. No. 5,053,121, which are incorporated herein by reference, U.S. Provisional Patent Application Ser. No. 61/451,903 and PCT/US2012/028522, also previously incorporated herein by reference.

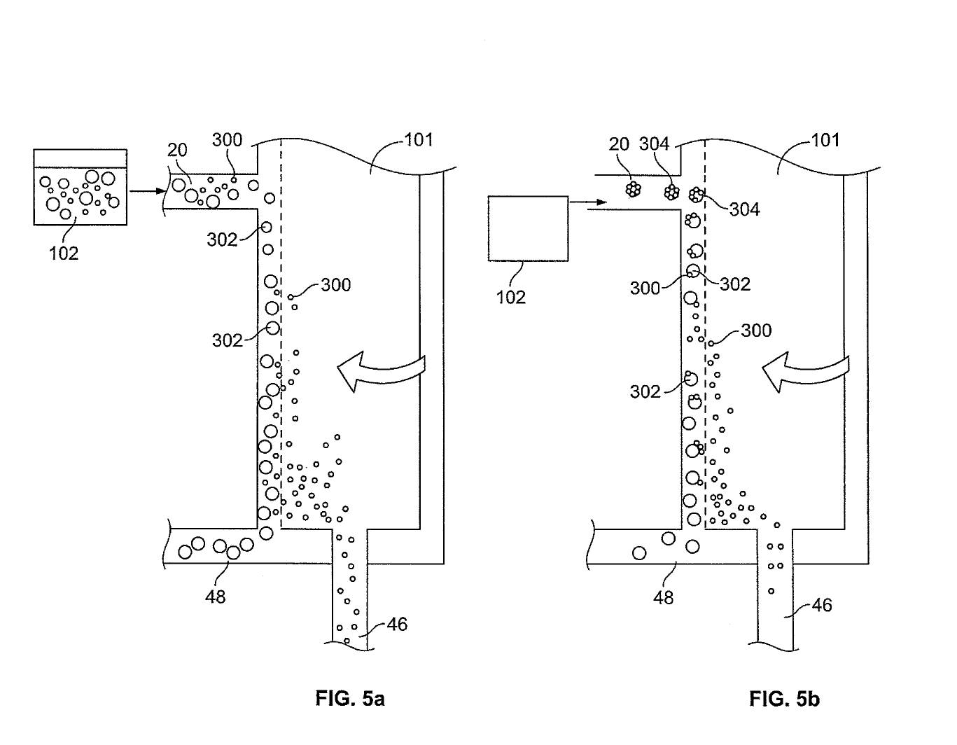

As shown in FIG. 1 (and described in greater detail in connection with FIGS. 3, 5(a)-5(b)), the spinning membrane separator 101 has at least two outlet ports. Outlet 46 of separator 101 receives the separated filtrate (e.g., separated biological cells) and is connected to tubing 138, which defines a flow path to filtrate/cell container 140. The filtrate/cell container may further include connection port 141 for sampling the contents within the filtrate/cell container 140.

Separation device 101 preferably includes a second outlet 48 that is connected to tubing segment 142 for directing the microcarriers to branched-connector 144, which branches into and defines a flow path to one or more in-process containers 122 and/or a flow path to a retentate container 150.

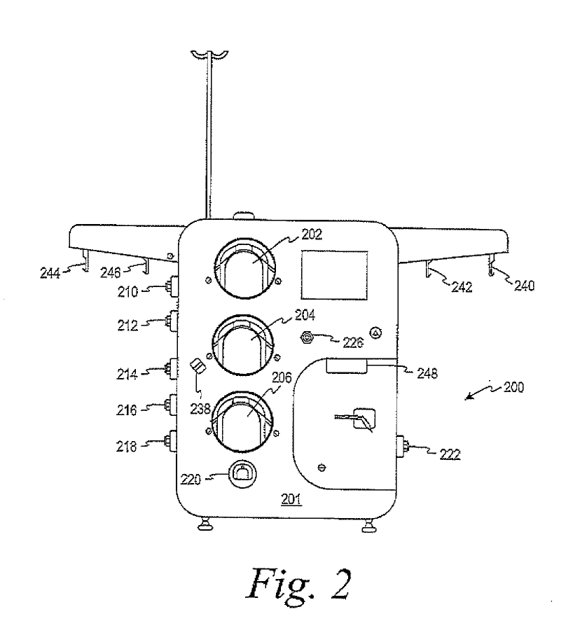

FIG. 2 shows the front panel 201 of reusable hardware processing apparatus 200. Apparatus 200 may be of compact size suitable for placement on a table top of a lab bench and adapted for easy transport. Alternatively, apparatus 200 may be supported by a pedestal that can be wheeled to its desired location. In any event, as shown in FIG. 2, apparatus 200 includes a plurality of peristaltic pumps, such as pumps 202, 204 and 206 on front panel 201. Pump segments of the disposable fluid circuit (described above) are selectively associated with peristaltic pumps 202, 204, and 206. The peristaltic pumps articulate with the fluid sets of FIG. 1 at the pump segments identified by reference numerals 162, 166, 168 and advance the cell suspension or other fluid within the disposable set, as will be understood by those of skill in the art. Apparatus 200 also includes clamps 210, 212, 214, 216, and 218. Clamps 210, 212, 214, 216 and 218 are used to control the flow of the cell suspension through different segments of the disposable set, as described above.

Apparatus 200 also includes several sensors to measure various conditions. The output of the sensors is utilized by device 200 to operate one or more processing or wash cycles. One or more pressure transducer sensor(s) 226 may be provided on apparatus 200 and may be associated with a disposable set 100 at certain points to monitor the pressure during a procedure. Pressure transducer 226 may be integrated into an in-line pressure monitoring site (at, for example, tubing segment 136), to monitor pressure inside separator 101. Air detector sensor 238 may also be associated with the disposable set 100, as necessary. Air detector 238 is optional and may be provided to detect the location of fluid/air interfaces.

Apparatus 200 includes weight scales 240, 242, 244, and 246 from which the cell container, in-process container, source container, and any additional container(s) (e.g., wash solution container, cleaving agent container, retentate container), respectively, may depend and be weighed. The weights of the bags are monitored by weight sensors and recorded during a washing or other procedure. From measurements of the weight sensors, the device, under the direction of the controller, determines whether each container is empty, partially full, or full and controls the components of apparatus 200, such as the peristaltic pumps and clamps 210, 212, 214, 216, 218, 220, 222, and 224.

Apparatus 200 includes at least one drive unit or “spinner” 248 (FIG. 4), which causes the indirect driving of the spinning membrane separator 101. Spinner 248 may consist of a drive motor connected and operated by apparatus 200, coupled to turn an annular magnetic drive member including at least a pair of permanent magnets. As the annular drive member is rotated, magnetic attraction between corresponding magnets within the housing of the spinning membrane separator cause the spinner within the housing of the spinning membrane separator to rotate.

Turning to FIG. 3, a spinning membrane separation device, generally designated 101, is shown. Such a device 101 forms part of the disposable circuit 100. Device 101 includes a generally cylindrical housing 12, mounted concentrically about a longitudinal vertical central axis. An internal member 14 is mounted concentric with the central axis 11. Housing 12 and internal member 14 are relatively rotatable. In the preferred embodiment, as illustrated, housing 12 is stationary and internal member 14 is a rotating spinner that is rotatable concentrically within cylindrical housing 12, as shown by the thick arrow in FIG. 3. The boundaries of the blood flow path are generally defined by gap 16 between the interior surface of housing 12 and the exterior surface of rotary spinner 14. The spacing between the housing and the spinner is sometimes referred to as the shear gap. In one non-limiting example, the shear gap may be approximately 0.025-0.050 inches (0.067-0.127 cm) and may be of a uniform dimension along axis 11, for example, where the axis of the spinner and housing are coincident. The shear gap may also vary circumferentially for example, where the axis of the housing and spinner are offset.

The shear gap also may vary along the axial direction, for example preferably an increasing gap width in the direction of flow. Such a gap width may range from about 0.025 to about 0.075 inches (0.06-0.19 cm). The gap width could be varied by varying the outer diameter of the rotor and/or the inner diameter of the facing housing surface. The gap width could change linearly or stepwise or in some other manner as may be desired. In any event, the width dimension of the gap is preferably selected so that at the desired relative rotational speed, Taylor-Couette flow, such as Taylor vortices, are created in the gap.

In accordance with the present disclosure, the biological cell/microcarrier suspension is fed from an inlet conduit 20 through an inlet orifice 22, which directs the fluid into the fluid flow entrance region in a path tangential to the circumference about the upper end of the spinner 14. At the bottom end of the cylindrical housing 12, the housing inner wall includes an exit orifice 48.

In the illustrated embodiment, the surface of the rotary spinner 14 is at least partially, and is preferably substantially or entirely, covered by a cylindrical porous membrane 62. The membrane 62 typically has a nominal pore size sufficient to exclude the microcarriers while allowing the biological cells to pass through. In one embodiment, membrane 62 typically has a nominal pore size of approximately 20 μm-50 μm, and more preferably approximately 30 μm, but other pore sizes may alternatively be used.

Of course, the pore size of membrane will be determined by the diameters of the microcarriers and adherent cells. The pores of membrane 62 should be sized to prevent passage of the microcarriers 302 (FIGS. 5a and 5b) but allow the desired cells to pass through the membrane. A typical diameter for microcarriers is 90-220 μm.

Membranes useful in the methods described herein may be fibrous mesh membranes, cast membranes, track-etched membranes or other types of membranes that will be known to those of skill in the art. For example, in one embodiment, the membrane may be made of a thin (approximately 10-15 μm thick) sheet of, for example, polycarbonate. In this embodiment, pores (holes) may be cylindrical and larger than those described above. For example, pores may be approximately 20-50 μm and more preferably about 30 μm. As noted above, the pores may be sized to allow the desired biological cells (e.g., white blood cells, red blood cells, stem cells) to pass, while the carriers (e.g., microcarriers) are retained within gap 16 for removal from separator 101.

Many of the steps of the method of processing disclosed herein are performed by a software driven microprocessing unit or “controller” of apparatus 200, with certain steps performed by the operator, as noted. For example, the apparatus 200 is switched on, and conducts self-calibration checks, including the checking of the peristaltic pumps, clamps, and sensors. Apparatus 200 under the direction of the controller then prompts the user to enter selected procedural parameters, such as the processing procedure to be performed, the amount of cell suspension to be processed, the number of cycles to take place, etc. The operator may then select and enter the procedural parameters for the separation procedure. The controller may also direct or effect other steps of the method which will now be described.

In accordance with the present disclosure, a method for separating biological cells from microcarriers or other substrate on which the cells have been grown is provided. The method utilizes the disposable fluid circuit 100 of the type described above, mounted on the reusable processing apparatus 200. After the circuit 100 is mounted, a source of biological cells and microcarriers is provided to the circuit. In one embodiment, the source (i.e., suspension) of biological cells and microcarriers may be transferred from a container in which the cells have been grown or in which the cells have been cleaved from the microcarriers. In another embodiment, the biological cells and microcarriers may be provided from a container wherein the cells and microcarriers have been drained after cleaving. In any event, the biological cells and microcarriers may be transferred, in sterile fashion, to source container 102 of circuit 100. Alternatively, source container 102 with the suspension of biological cells and microcarriers already contained therein may be directly attached, likewise in sterile fashion, to circuit 100. Thus, the source of the suspension to be processed will include at least the biological cells and microcarriers. The suspension may further include some of the culture media in which the cells were grown and/or cleaving agent used to effect cleaving of the cells from the microcarrier. Once circuit 100 has been loaded onto apparatus 200, the system will initiate and undergo system checks to ensure proper loading of the circuit 100.

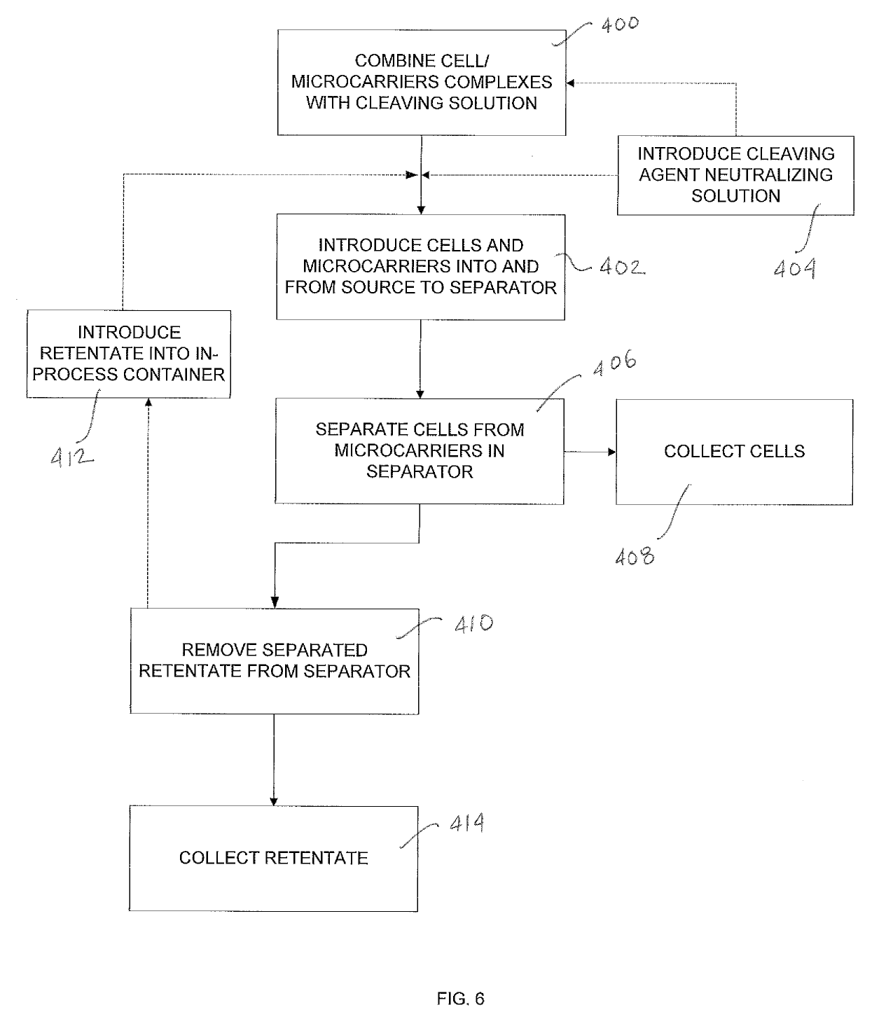

In one embodiment, if not already cleaved, the biological cells are cleaved from the microcarriers prior to introduction into circuit 100 (FIG. 6, step 400). The system may be programmed to deliver a selected amount of a cleaving solution from a container (not shown) connected to circuit 100 at one of access sites 134a or 134b. A selected period of time may be provided to allow effective cleaving to take place within container 102. A suitable cleaving solution may be the above-described Trypsin or other synthetic alternative. Prior to introduction of biological cells and microcarriers into separator 101, the system (under the direction of the controller) may initiate a priming of the flow paths of the circuit. The circuit may be primed with a priming solution, such as wash solution, a cleaving solution or other solution such as the cleaving agent neutralizing solution suspended from one more hangers/weight scales of the reusable separation apparatus.

After the system has been primed, the controller directs one or more peristaltic pumps to rotate and draw the suspension from source container 102 through the flow paths of circuit 100. The suspension of biological cells and microcarriers is introduced into separator 101 through inlet 20 where it enters gap 16 (FIG. 6, step 402). If the concentration of biological cells and microcarriers is too high, the suspension of cells and microcarriers may be diluted with saline, additional cleaving agent, the cleaving agent neutralizing solution or other solution. Under the influence of the forces (e.g., Taylor vortices) generated by the spinning action of separator 101, the larger microcarriers are separated from the biological cells. Biological cells 300 (see FIG. 5a) are able to pass through the membrane 62 and into the interior of separator 101 from which they exit via outlet 46 (FIG. 6, step 406). The biological cells flow through flow path 138 and may be collected in cell/filtrate bag 140 for further processing or treatment (FIG. 6, step 408). For example, the now separated and collected cells may provide the source of biological cells that are processed in accordance with the methods and systems described in U.S. patent application Ser. No. 14/122,855, which is incorporated herein by reference. The remaining microcarriers are too large to pass through membrane 62 and exit gap 16 through outlet 48 together with any unseparated cell/carrier aggregates and any other suspension components that do not pass through membrane 62 (collectively, referred to as “retentate”). Retentate exits gap 16 of separator 101 and flows (under the influence of one or more pumps) to one or both of retentate bag 150 or in-process bag 122 (FIG. 6, step 412). As shown in FIG. 5(b), some of the cells 300 may still be attached to microcarriers 302 when entering gap 16. The spinning (agitating) action of separator 101 during initial introduction into gap 16 or in subsequent cycles of processing of the retentate from in-process container 122 may assist in separating the cells from the microcarriers.

In one embodiment, where the amount of collected cells (as measured by one of weight scales 240, 242, 244 or 246) is determined to be low, the controller will direct flow of the suspension through outlet 48 to in-process container 122. The suspension of microcarriers and residual biological cells may then be withdrawn/pumped from in-process container 122 to separator 101 and, optionally, combined with additional wash solution such as a cleaving agent neutralizing solution, for one or more additional separation cycles, as needed.

As indicated above, a cleaving agent neutralizing solution may be added to the suspension before or during processing (FIG. 6, step 404). Cleaving agent neutralizing agent may be supplied from a container attached in sterile fashion to source container 102 prior to processing. At one of access sites 134a or 135b under the direction of the controller, a selected volume of cleaving agent neutralizing solution may be metered (e.g., pumped) into source container 102 or line 136 where it is combined with the suspension of cells and microcarriers.

As noted above, in accordance with the method of the present disclosure, the suspension of biological cells may be diluted (or further diluted) to avoid introducing too many of the microparticles and cells into gap 16 of separator 101, which may affect (negatively) the efficiency of the separation. Thus, the system may be programmed to determine a Total Cell Volume (TCV) % for the incoming stream of cells and microcarriers. The TCV % may be determined by the following equation:

$$TCV \% = \frac{Total Volume of Cells + Total Volume of Microcarriers}{Total Volume of Suspension}$$

A suitable TCV may depend in part on the rotational speed of the spinning membrane 101, the filtrate flux, the size and concentration and the membrane pore size. To arrive at the desired TCV % for a given procedure, the system, under the direction of the controller, delivers the required volume of liquid from the container 135a of wash or other solution suspended from one of weight scales 240, 242, 244 or 246.

The description provided above is intended for illustrative purposes, and is not intended to limit the scope of the disclosure to any particular method, system, apparatus or device described herein.

1. A method for separating biological cells from microcarriers in a suspension comprising:
a) introducing a suspension of biological cells and microcarriers into a separation device comprising a relatively rotatable cylindrical housing and an internal member, wherein said cylindrical housing has an interior surface and said internal member has an exterior surface, said surfaces defining a gap therebetween, wherein one of said surfaces includes a porous membrane comprising pores sized to retain said microcarriers while allowing said biological cells to pass through said membrane; and
b) withdrawing said biological cells from said separation device.

2. The method of claim 1 wherein said pore size is less than or equal to approximately 50 μm.

3. The method of claim 2 wherein said pore size is greater than or equal to approximately 20 μm.

4. The method of claim 1 comprising introducing said suspension of biological cells into said gap.

5. The method of claim 1 comprising introducing said suspension of biological cells from a container that is in fluid communication with said gap.

6. The method of claim 1 wherein said biological cells are introduced through an inlet in flow communication with said gap and said microcarriers are removed through an outlet in flow communication with said gap.

7. The method of claim 1 wherein said biological cells are adhered to the surface of said microcarriers during said introducing step.

8. The method of claim 5 further comprising introducing a cleaving agent into said container prior to introducing said biological cells into said separator.

9. The method of claim 1 wherein said microcarriers comprise polymeric, coated beads.

10. The method of claim 1 wherein said membrane is made of a material selected from the group of nylon and polycarbonate.

11. The method of claim 1 further comprising determining the amount of microcarriers and biological cells that are introduced into said separation device.

12. The method of claim 11 comprising determining the Total Packed Volume (TCV) Percentage of said suspension being introduced into said separation device.

13. The method of claim 12 wherein said Total Packed Volume (TCV) Percentage is determined by:
TCV %=Total Volume of Cells+Total Volume of Microcarriers/Total Volume of Suspension

14. The method of claim 1 further comprising combining said suspension with a cleaving agent neutralizing solution.

15. The method of claim 1 wherein said biological cells are selected from the group of white blood cells, red blood cells and stem cells.

16. A system for separating biological cells from microcarriers in a suspension comprising:
a) a reusable hardware unit comprising a separation device drive unit for receiving a separation device;
b) a disposable fluid circuit mountable on said reusable hardware unit, said disposable fluid circuit including a separation device comprising a relatively rotatable cylindrical housing and an internal member, wherein said cylindrical housing has an interior surface and said internal member has an exterior surface, said surfaces defining a gap therebetween, wherein one of said surfaces includes a porous membrane comprising pores sized to retain said microcarriers while allowing said biological cells to pass through said membrane, said separation device including an inlet and at least one outlet in fluid communication with said gap; and
c) a controller configured and/or programmed to control the operation of said separation device.

17. The system of claim 16 wherein said controller configured and/or programmed to determine the Total Packed Volume % in said suspension.

18. The system of claim 17 wherein said controller is configured and/or programmed to determine a volume of a suspension of biological cells and microcarriers to be introduced into said separation device.

19. The system of claim 16 wherein said porous membrane has a pore size of approximately 20 μm-50 μm.

20. The system of claim 19 wherein said porous membrane has a pore size of approximately 30 μm.
